(19) United States
(12) Patent Application Publication
(10) Pub. No.: US 2004/0176448 A1
(43) Pub. Date: Sep. 9, 2004

(54) Use of carnitine and/or one or more acyl-carnitines for producing cosmetic or dermatological preparations, which increase ceramide biosynthesis

(76) Inventors: Thomas Blatt, Wedel (DE); Thomas Doring, Hamburg (DE); Christopher Mummert, Bienenbuttel (DE); Claudia Mundt, Bremen (DE)

Correspondence Address: GREENBLUM & BERNSTEIN, P.L.C., 1950 ROLAND CLARKE PLACE, RESTON, VA, 20191, US

(21) Appl. No.: 10/480,261

(22) PCT Filed: Jun. 7, 2002
(86) PCT No.: PCT/EP02/06254

(30) Foreign Application Priority Data
Jun. 19, 2001 (DE) 101 29 502.2

(51) Int. Cl. A61K 31/225; A61K 31/198

(52) U.S. Cl. 514/547; 514/565

(57) ABSTRACT

The use of carnitine and/or one or more acylcarnitines for producing cosmetic or dermatological preparations for producing cosmetic or dermatological preparations for increasing the biosynthesis of ceramide.

The present invention relates to cosmetic and dermatological preparations comprising active ingredients for the care and for the protection of the skin, in particular of sensitive skin, and especially of skin aged or aging by intrinsic and/or extrinsic factors, and to the use of such active ingredients and combinations of such active ingredients in the field of cosmetic and dermatological skincare.

Cosmetic skincare is primarily understood as meaning that the natural function of the skin as a barrier against environmental influences (e.g. dirt, chemicals, microorganisms) and against the loss of substances intrinsic to the body (e.g. water, natural fats, electrolytes) is strengthened or restored.

Impairment of this function may lead to increased resorption of toxic or allergenic substances or to attack by microorganisms, leading to toxic or allergic skin reactions.

In the case of aged skin, for example, regenerative renewal takes place at a slower rate, where, in particular, the water-binding capacity of the horny layer deteriorates. It therefore becomes inflexible, dry and chapped (“physiologically” dry skin). Barrier damage is the result. The skin becomes susceptible to negative environmental influences, such as the invasion of microorganisms, toxins and allergens. This may even result in toxic or allergic skin reactions.

In the case of pathologically dry and sensitive skin, barrier damage is present a priori. Epidermal intercellular lipids become defective or are formed in an inadequate amount or composition. The consequence is increased permeability of the horny layer and inadequate protection of the skin against the loss of hygroscopic substances and water.

The barrier effect of the skin can be quantified via the determination of the transepidermal water loss (TEWL). This is the evaporation of water from inside the body without taking into account the loss of water during perspiration. Determination of the TEWL value has proven to be extraordinarily informative and can be used to diagnose chapped or cracked skin, for determining the compatibility of surfactants which have very different chemical structures, and more besides.

For the beauty and well-cared-for appearance of the skin, the proportion of water in the uppermost layer of the skin is of greatest significance. It can be favorably influenced within a limited scope by introducing moisture regulators.

Anionic surfactants, which are generally constituents of cleansing preparations, can increase the pH in the horny layer with lasting effect, which severely hinders regenerative processes which serve to restore and renew the barrier function of the skin. In this case, a new, frequently very unfavorable state of equilibrium is established in the horny layer between regeneration and the loss of essential substances as a result of regular extraction; this state has a decisive adverse effect on the external appearance of the skin and the physiological mode of function of the horny layer.

Even simple bathing in water without the addition of surfactants will initially cause the horny layer of the skin to swell, the degree of this swelling depending, for example, on the bathing time and its temperature. As well as water-soluble substances, e.g. water-soluble constituents of dirt, substances which are endogenous to the skin which are responsible for the water-binding capacity of the horny layer are also washed off or out. In addition, as a result of surface-active substances endogenous to the skin, fats in the skin are also dissolved and washed out to a certain extent. After the initial swelling, this causes a subsequent significant drying-out of the skin, which may be further intensified by washing-active additives.

In healthy skin these processes are generally of no consequence since the protective mechanisms of the skin can readily compensate for such slight disturbances to the upper layers of the skin. However, even in the case of nonpathological deviations from the norm, e.g. as a result of wear damage or irritations caused by the environment, photodamage, aging skin etc., the protective mechanism of the surface of the skin is impaired. In some circumstances it is then no longer able to fulfill its role by itself and has to be regenerated by external measures.

Moreover, it is known that the lipid composition and amount of the horny layer of pathologically altered, dry and dry but not diseased skin of younger and older people deviates from the normal state found in the healthy normally hydrated skin of a group of the same age. In this connection, the changes in the lipid pattern of very dry, noneczematous skin of patients with atopic eczema represents an extreme case of the deviations which are found in the dry skin of people with healthy skin.

Here, these deviations affect very particularly the ceramides, which are severely reduced in number and additionally have a different composition. Here, the deficit of ceramides 1 and 3 is particularly striking, it being known for ceramide 1 in particular that it increases in a particular way the order of the lipids in the intercellular membrane systems.

Adverse changes in the lipid membranes of the type described above are possibly based on incorrectly controlled lipid biosynthesis and in the end effect likewise increase transepidermal water loss. In turn, permanent barrier weakening makes skin which is itself healthy more sensitive and can in certain instances contribute to the appearance of eczematous processes in diseased skin.

The effect of ointments and creams on barrier function and hydration of the horny layer usually does not consist in the rebuilding or strengthening of the physical-chemical properties of the lamellae of intercellular lipids. An essential partial effect is based on the mere coverage of the areas of skin treated and the blockage of water resulting therefrom in the horny layer lying below. Co-applied hygroscopic substances bind the water, resulting in a measurable increase in the water content in the horny layer. However, this purely physical barrier can be removed again relatively easily. After use of the product is stopped, the skin then reverts very quickly to the state prior to the start of treatment. Moreover, the skincare effect can decrease upon regular treatment, meaning that ultimately the status quo is again achieved even during treatment. In the case of certain products, the condition of the skin deteriorates temporarily in some circumstances when use is stopped. A permanent product effect is therefore as a rule not achieved or achieved only to a limited extent.

In order to aid deficient skin in its natural regeneration and to strengthen its physiological function, intercellular lipid mixtures have recently increasingly been added to topical preparations which are intended to be used by the skin to rebuild the natural barrier. However, these lipids, but in particular the ceramides, are very expensive raw materials. In addition, their effect is in most cases very much lower than hoped for.

The aim of the present invention was therefore to find ways to avoid the disadvantages of the prior art. In particular, the effect of skincare products should be physiological, rapid and long-lasting.

For the purposes of the present invention, skincare is understood primarily as meaning that the natural function of the skin as a barrier against environmental influences (e.g. dirt, chemicals, microorganisms) and against the loss of substances endogenous to the body (e.g. water, lipids, electrolytes) is strengthened or restored.

Products for the care, treatment and cleansing of dry and stripped skin are known per se. However, their contribution to the regeneration of a physiologically intact, hydrated and smooth horny layer is limited with regard to extent and time.

The effect of ointments and creams on the barrier function and the hydration of the horny layer is based essentially on the coverage (occlusion) of the areas of skin treated. The ointment or cream represents, as it were, a (second) artificial barrier which is intended to prevent loss of water by the skin. It is equally easy to remove this physical barrier again, for example using cleansers, as a result of which the original, impaired state is again achieved. Moreover, the skincare effect can decrease upon regular treatment. After use of the product is stopped, the skin reverts very quickly to the state prior to the start of treatment. In the case of certain products, the condition of the skin is even temporarily worsened in some circumstances. A long-lasting product effect is therefore generally not achieved or is achieved only to a limited extent.

The effect of some pharmaceutical preparations on the barrier function of the skin consists even in selective damage to the barrier, which is intended to make it possible for active ingredients to be able to penetrate into or through the skin into the body. Here, a disturbed appearance of the skin as a side-effect is accepted to some extent as a small price to pay.

The effect of caring cleansing products consists essentially in an efficient refatting with sebum lipid-like substances. The simultaneous reduction in the surfactant content of such preparations permits a further limitation of the damage to the horny layer barrier.

However, the prior art lacks preparations which have a positive influence on the barrier function and hydration of the horny layer and enhance or even restore the physicochemical properties of the horny layer and, in particular, of the lamellae comprising intercellular lipids.

The object of the present invention was therefore to overcome the disadvantages of the prior art. In particular, the aim was to provide skincare preparations and preparations for cleansing the skin which retain or restore the barrier properties of the skin, especially when the natural regeneration of the skin is inadequate. In addition, they should be suitable for the treatment and prophylaxis of damage caused by the skin drying out, for example fissures or inflammatory or allergic processes, and also neurodermitis. The object of the present invention was also to provide stable skincare cosmetic and/or dermatological compositions which protect the skin against environmental influences such as sun and wind. In particular, the effect of the preparations should be physiological, rapid and long-lasting.

According to the invention, the shortcomings of the prior art are overcome by the use of carnitine and/or one or more acylcarnitines for producing cosmetic or dermatological preparations for producing cosmetic or dermatological preparations for increasing the biosynthesis of ceramide, for improving the barrier properties of the skin, and for preventing or reducing skin drying.

The active ingredient combinations according to the invention or cosmetic or dermatological preparations comprising such active ingredient combinations are entirely satisfactory preparations in every respect. It could not have been foreseen by the person skilled in the art that the preparations according to the invention

better retain or restore the barrier properties of the skin,

better counteract skin drying,

better protect the skin against environmental influences than the preparations of the prior art.

The use of acylated and nonacylated carnitine in cosmetic or dermatological preparations is known per se. For example, FR-A 2 654 618 describes the use of L-carnitine derivatives in cosmetic preparations for regulating cell growth. U.S. Pat. No. 4,839,159 describes topical preparations for improving or preventing harmful skin states, including wrinkling, which is attributed to a loss of elasticity in the skin.

L-Carnitine[3-hydroxy-4-(trimethylammonio)butyric acid betain] has the structural formula

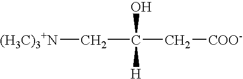

(empirical formula C₇H₁₅NO₃).

The L form of carnitine is widespread in animal tissue, in particular striated muscle. In the fatty acid metabolism, it serves as a transporter for acyl groups through the mitochondrial membrane. These are transported by an acyl transferase by acyl-coenzyme A to the hydroxy group of the L-carnitine. The transport of L-carnitine and acyl-L-carnitine through the membrane takes place by mediation of a transport protein (translocase). Both enantiomers (D and L form) are advantageous for use for the purposes of the present invention. It may also be advantageous to use any desired enantiomer mixtures, for example a racemate of D and L form.

According to the invention, acylcarnitines are chosen from the group of substances of the following general structural formula

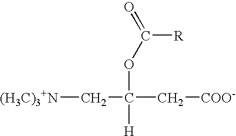

where R is chosen from the group of branched and unbranched alkyl radicals having up to 10 carbon atoms. Preference is given to propionyl carnitine and very particular preference is given to acetyl carnitine. Both enantiomers (D and L form) are advantageous for use for the purposes of the present invention. It may also be advantageous here to use any desired enantiomer mixtures, for example a racemate of D and L form.

Advantageously, the preparations according to the invention comprise 0.001-10% by weight of carnitine and/or one or more acylcarnitines, based on the total weight of the preparations.

According to the invention, it is, in particular, extremely advantageous to use the active ingredient or ingredients used according to the invention or cosmetic or topical dermatological preparations with an effective content of active ingredient combination used according to the invention for the cosmetic or dermatological treatment or prophylaxis of undesired skin conditions.

According to the invention, customary antioxidants can be used to preparations which comprise the active ingredient combinations according to the invention.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, alaninediacetic acid, flavonoids, polyphenols, catechins, vitamin C and derivatives (e.g. ascorbyl palmitate, Mg ascorbyl phosphate, ascorbyl acetate), tocopherols and derivatives (e.g. vitamin E acetate), and coniferyl benzoate of benzoin resin, rutinic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of these said active ingredients which are suitable according to the invention.

The amount of antioxidants (one or more compounds) in the preparations is preferably 0.001 to 30% by weight, particularly preferably 0.05-20% by weight, in particular 1-10% by weight, based on the total weight of the preparation.

The prophylaxis or the cosmetic or dermatological treatment with the active ingredient used according to the invention or with the cosmetic or topical dermatological preparations with an active content of active ingredient used according to the invention is carried out in the usual manner, by applying the active ingredient used according to the invention or the cosmetic or topical dermatological preparations with an active content of active ingredient used according to the invention to the affected areas of skin.

The active ingredient used according to the invention can advantageously be incorporated into customary cosmetic and dermatological preparations, which may be in various forms. Thus, they may, for example, be a solution, an emulsion of the water-in-oil (W/O) type or of the oil-in-water (O/W) type, or a multiple emulsions, for example of the water-in-oil-in-water (W/O/W) type or oil-in-water-in-oil (O/W/O) type, a hydrodispersion or lipodispersion, a gel, a solid stick or an aerosol.

Emulsions according to the invention for the purposes of the present invention, e.g. in the form of a cream, a lotion, a cosmetic milk, are advantageous and comprise, for example, fats, oils, waxes and/or other fatty substances, and water and one or more emulsifiers as are customarily used for this type of formulation.

It is also possible and advantageous for the purposes of the present invention to incorporate the active ingredient used according to the invention into aqueous systems or surfactant preparations for cleansing the skin and the hair.

The person skilled in the art is of course aware that demanding cosmetic compositions are mostly inconceivable without the customary auxiliaries and additives. The cosmetic preparations according to the invention can therefore comprise cosmetic auxiliaries, as are customarily used in such preparations, e.g. preservatives, bactericides, deodorizing substances, antiperspirants, insect repellents, vitamins, antifoams, dyes, pigments with a coloring action, thickeners, softening substances, moisturizing substances and/or humectant substances, fats, oils, waxes or other customary constituents of a cosmetic formulation, such as alcohols, polyols, polymers, foam stabilizers, electrolytes, organic solvents or silicone derivatives.

Corresponding requirements apply mutatis mutandis to the formulation of medicinal preparations.

Medicinal topical compositions for the purposes of the present invention generally comprise one or more medicaments in an effective concentration. For the sake of simplicity, for a clear distinction between cosmetic and medicinal application and corresponding products, reference is made to the legal provisions of the Federal Republic of Germany (e.g. Cosmetics Directive, Foods and Drugs Act).

Preparations according to the invention can advantageously also comprise substances which absorb UV radiation in UVB range, where the total amount of the filter substances is, for example, 0.1% by weight to 30% by weight, preferably 0.5 to 10% by weight, in particular 1.0 to 6.0% by weight, based on the total weight of the preparations in order to provide cosmetic preparations which protect the hair or the skin from the entire range of ultraviolet radiation. They can also be used as sunscreens for hair.

If the preparations according to the invention comprise UVB filter substances, these may be oil-soluble or water-soluble. Examples of oil-soluble UVB filters which are advantageous according to the invention are:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate;

esters of cinnamic acid, preferably 2-ethylhexyl 4-methoxycinnamate, isopentyl 4-methoxycinnamate;

esters of salicylic acid, preferably 2-ethylhexyl salicylate, 4-isopropylbenzyl salicylate, homomenthyl salicylate,

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably di(2-ethylhexyl)4-methoxybenzalmalonate,

2,4,6-trianilino(p-carbo-2′-ethyl-1′-hexyloxy)-1,3,5-triazine.

Examples of advantageous water-soluble UVB filters are:

salts of 2-phenylbenzimidazole-5-sulfonic acid, such as its sodium, potassium or its tri-ethanolammonium salt, and the sulfonic acid itself;

sulfonic acid derivatives of benzophenones, preferably 2-hydroxy-4-methoxybenzo-phenone-5-sulfonic acid and its salts;

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and its salts, and 1,4-di(2-oxo-10-sulfo-3-bornylidenemethyl)benzene and its salts (the corresponding 10-sulfato compounds, for example the corresponding sodium, potassium or triethanolammonium salt), also referred to as benzene-1,4-di(2-oxo-3-bornylidenemethyl-10-sulfonic acid.

The list of specified UVB filters which can be used in combination with the active ingredient combinations according to the invention is of course not intended to be limiting.

It may also be advantageous to use UVA filters which are usually present in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropyl-phenyl)propane-1,3-dione. The amount used for the UVB combination may be used.

Cosmetic and dermatological preparations according to the invention advantageously also comprise inorganic pigments based on metal oxides and/or other metal compounds which are insoluble or sparingly soluble in water, in particular the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. The pigments are particularly preferably based on TiO₂.

For the purposes of the present invention, it is particularly advantageous, although not obligatory, for the inorganic pigments to be present in hydrophobic form, i.e. to have been treated on the surface to repel water. This surface-treatment may involve providing the pigments with a thin hydrophobic layer by processes known per se.

One such process involves, for example, producing the hydrophobic surface layer in accordance with a reaction according to

n TiO₂+m(RO)₃Si—R′→n TiO₂ (surf.)

Here, n and m are stoichiometric parameters to be used as desired, R and R′ are the desired organic radicals. For example, hydrophobicized pigments prepared analogously to DE-A 33 14 742 are advantageous.

Advantageous TiO₂ pigments are available, for example, under the trade names MT 100 T from TAYCA, and also M 160 from Kemira and T 805 from Degussa.

Preparations according to the invention may, especially when crystalline or microcrystalline solid bodies, for example inorganic micropigments, are to be incorporated into the preparations according to the invention, also comprise anionic, nonionic and/or amphoteric surfactants. Surfactants are amphiphilic substances which can dissolve organic, nonpolar substances in water.

The hydrophilic moieties of a surfactant molecule are mostly polar functional groups, for example —COO⁻, —OSO₃²⁻, —SO₃⁻, whereas the hydrophobic moieties are usually nonpolar hydrocarbon radicals. Surfactants are generally classified according to the type and charge of the hydrophilic molecular moiety. In this connection, it is possible to differentiate between four groups:

anionic surfactants,

cationic surfactants,

amphoteric surfactants and

nonionic surfactants.

Anionic surfactants usually have, as functional groups, carboxylate, sulfate or sulfonate groups. In aqueous solution, they form negatively charged organic ions in acidic or neutral medium. Cationic surfactants are characterized almost exclusively by the presence of a quaternary ammonium group. In aqueous solution, they form positively charged organic ions in acidic or neutral medium. Amphoteric surfactants contain both anionic and cationic groups and accordingly in aqueous solution exhibit the behavior of anionic or cationic surfactants depending on the pH. In strongly acidic medium, they have a positive charge, and in alkali medium a negative charge. By contrast, in the neutral pH range, they are zwitterionic, as the example below is intended to illustrate:

| | | |
|---|---|---|
| RNH₂⁺CH₂CH₂COOH X⁻ | (at pH = 2) | X⁻ = any anion, e.g. Cl⁻ |
| RNH₂⁺CH₂CH₂COO⁻ | (at pH = 7) | |
| RNHCH₂CH₂COO⁻ B⁺ | (at pH = 12) | B⁺ = any cation, e.g. Na⁺ |

Typical nonionic surfactants are polyether chains. Nonionic surfactants do not form ions in aqueous medium.

A. Anionic Surfactants

Anionic surfactants which can be used advantageously are acylamino acids (and salts thereof), such as

1. acyl glutamates, for example sodium acyl glutamate, di-TEA-palmitoyl aspartate and sodium caprylic/capric glutamate,

2. acylpeptides, for example palmitoyl-hydrolyzed milk protein, sodium cocoyl-hydrolyzed soya protein and sodium/potassium cocoyl-hydrolyzed collagen,

3. sarcosinates, for example myristoyl sarcosine, TEA-lauroyl sarcosinate, sodium lauroyl sarcosinate and sodium cocoyl sarcosinate,

4. taurates, for example sodium lauroyl taurate and sodium methyl cocoyl taurate,

5. acyl lactylates, lauroyl lactylate, caproyl lactylate

6. alaninates

carboxylic acids and derivatives, such as

1. carboxylic acids, for example lauric acid, aluminum stearate, magnesium alkanolate and zinc undecylenate,

2. ester carboxylic acids, for example calcium stearoyl lactylate, laureth-6 citrate and sodium PEG-4 lauramide carboxylate,

3. ether carboxylic acids, for example sodium laureth-13 carboxylate and sodium PEG-6 cocamide carboxylate,

phosphoric esters and salts, such as, for example, DEA-oleth-10 phosphate and dilaureth-4 phosphate,

sulfonic acids and salts, such as

1. acyl isethionates, e.g. sodium/ammonium cocoyl isethionate,

2. alkylarylsulfonates,

3. alkylsulfonates, for example sodium cocomonoglyceride sulfate, sodium C₁₂₋₁₄-olefinsulfonate, sodium lauryl sulfoacetate and magnesium PEG-3 cocamide sulfate,

4. sulfosuccinates, for example dioctyl sodium sulfosuccinate, disodium laureth sulfosuccinate, disodium lauryl sulfosuccinate and disodium undecyleneamido-MEA sulfosuccinate

and

sulfuric esters, such as

1. alkyl ether sulfate, for example sodium, ammonium, magnesium, MIPA, TIPA laureth sulfate, sodium myreth sulfate and sodium C₁₂₋₁₃ parethsulfate,

2. alkyl sulfates, for example sodium, ammonium and TEA lauryl sulfate.

B. Cationic Surfactants

Cationic surfactants which can be used advantageously are

1. alkylamines,

2. alkylimidazoles,

3. ethoxylated amines and

4. quaternary surfactants

5. ester quats

Quaternary surfactants comprise at least one N atom which is covalently bonded to 4 alkyl and/or aryl groups. Irrespective of the pH, this leads to a positive charge. Alkylbetaine, alkylamidopropylbetaine and alkylamidopropylhydroxysulfain are advantageous quaternary surfactants. The cationic surfactants used according to the invention can also be preferably chosen from the group of quaternary ammonium compounds, in particular benzyltrialkylammonium chlorides or bromides, such as, for example, benzyldimethylstearylammonium chloride, and also alkyltrialkylammonium salts, for example for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example lauryl- or cetylpyrimidinium chloride, imidazoline derivatives and compounds with a cationic character, such as amine oxides, for example alkyl dimethylamine oxides or alkylaminoethyldimethylamine oxides. In particular, the use of cetyltrimethylammonium salts is advantageous.

C. Amphoteric Surfactants

Amphoteric surfactants which can be used advantageously are

1. acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate,

2. N-alkylamino acids, for example aminopropylalkylglutamide, alkylaminopropionic acid, sodium alkylimidodipropionate and lauroamphocarboxyglycinate.

D. Nonionic Surfactants

Nonionic surfactants which can be used advantageously are

1. alcohols,

2. alkanolamides, such as cocamides MEA/DEA/MIPA,

3. amine oxides, such as cocoamidopropylamine oxide,

4. esters which are formed by esterification of carboxylic acids with ethylene oxide, glycerol, sorbitan or other alcohols,

5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and cocoglycoside

6. sucrose esters, sucrose ethers

7. polyglycerol esters, diglycerol esters, monoglycerol esters

8. methyl glucose esters, esters of hydroxy acids

Also advantageous is the use of a combination of anionic and/or amphoteric surfactants with one or more nonionic surfactants.

The surface-active substance may be present in the preparations according to the invention in a concentration between 1 and 95% by weight, based on the total weight of the preparations.

The lipid phase of the cosmetic or dermatological emulsions according to the invention can advantageously be chosen from the following group of substances:

mineral oils, mineral waxes

oils, such as triglycerides of capric or of caprylic acid, and also natural oils such as, for example, castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alkyl benzoates;

silicone oils, such as dimethylpolysiloxanes, diethylpolysiloxanes, diphenylpolysiloxanes and mixed forms thereof.

The oil phase of the emulsions of the present invention is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, of silicone oils, of dialkyl ethers, the group of saturated or unsaturated, branched or unbranched alcohols, and the fatty acid triglycerides, namely the triglycerol esters of saturated and/or unsaturated, branched and/or unbranched alkanecarboxylic acids having a chain length of from 8 to 24, in particular 12-18 carbon atoms. The fatty acid triglycerides can, for example, advantageously be chosen from the group of synthetic, semisynthetic and natural oils, e.g. olive oil, sunflower oil, soybean oil, groundnut oil, rapeseed oil, almond oil, palm oil, coconut oil, palm kernel oil and the like.

Any mixtures of such oil and wax components can also be used advantageously for the purposes of the present invention. It may also in some instances be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Particularly advantageous mixtures are those of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate.

Of the hydrocarbons, paraffin oil, squalane and squalene are to be used advantageously for the purposes of the present invention.

The oil phase can advantageously also have a content of cyclic or linear silicone oils, or consist entirely of such oils, although it is preferable to use an additional content of other oil phase components apart from the silicone oil or the silicone oils. Such silicones or silicone oils may be in the form of monomers, which are generally characterized by structural elements, as follows:

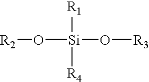

Linear silicones having two or more siloxyl units which are to be used advantageously according to the invention are generally characterized by structural elements, as follows:

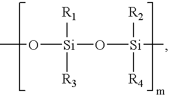

where the silicon atoms can be substituted by identical or different alkyl radicals and/or aryl radicals, which are shown here in general terms by the radicals R₁-R₄ (that is to say the number of different radicals is not necessarily limited to 4). m can assume values from 2-200 000.

Cyclic silicones to be used advantageously according to the invention are generally characterized by structural elements, as follows

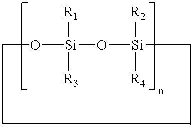

where the silicon atoms can be substituted by identical or different alkyl radicals and/or aryl radicals, which are shown here in general terms by the radicals R₁-R₄ (that is to say the number of different radicals is not necessarily limited to 4). n can assume values from 3/2 to 20. Fractions for n take into consideration that uneven numbers of siloxyl groups may be present in the cycle.

Advantageously, cyclomethicone (e.g. decamethylcyclopentasiloxane) is used as the silicone oil to be used according to the invention. However, other silicone oils are also to be used advantageously for the purpose of the present invention, for example undecamethylcyclotrisiloxane, polydimethylsiloxane, poly(methylphenylsiloxane), cetyldimethicone, behenoxydimethicone.

Also advantageous are mixtures of cyclomethicone and isotridecyl isononanoate, and those of cyclomethicone and 2-ethylhexyl isostearate.

It is, however, also advantageous to choose silicone oils of similar constitution to the above-described compounds whose organic side chains are derivatized, for example polyethoxylated and/or polypropoxylated. These include, for example, polysiloxane-polyalkyl-polyether copolymers, such as cetyl-dimethicone copolyol, (cetyl-dimethicone copolyol (and) polyglyceryl-4-isostearate (and) hexyl laurate).

Also particularly advantageous are mixtures of cyclomethicone and isotridecyl isononanoate, and of cyclomethicone and 2-ethylhexyl isostearate.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and, in particular, one or more thickeners which can advantageously be chosen from the group consisting of silicon dioxide and aluminum silicates.

Preparations according to the invention in the form of emulsions advantageously comprise, in particular, one or more hydrocolloids. These hydrocolloids can advantageously be chosen from the group of gums, polysaccharides, cellulose derivatives, phyllosilicates, polyacrylates and/or other polymers.

Preparations according to the invention in the form of hydrogels comprise one or more hydrocolloids. These hydrocolloids can advantageously be chosen from the abovementioned group.

The gums include saps from plants or trees which harden in the air and form resins, or extracts from aquatic plants. From this group, for the purposes of the present invention, gum arabic, carob flour, tragacanth, karaya, guar gum, pectin, gellan gum, carrageen, agar, algins, chondrus, xanthan gum, for example, can be chosen advantageously.

Also advantageous is the use of derivatized gums, such as, for example, hydroxypropyl guar (Jaguar® HP 8).

The polysaccharides and polysaccharide derivatives include, for example, hyaluronic acid, chitin and chitosan, chondroitin sulfates, starch and starch derivatives.

The cellulose derivatives include, for example, methylcellulose, carboxymethylcellulose, hydroxyethylcellulose, hydroxypropylmethylcellulose.

The phyllosilicates include naturally occurring and synthetic clay earths, such as, for example, montmorillonite, bentonite, hectorite, laponite, magnesium aluminum silicates such as Veegum®. These can be used as such or in modified form, such as, for example, stearylalkonium hectorites.

In addition, silica gels can also be used advantageously.

The polyacrylates include, for example, Carbopol grades from Goodrich (Carbopol 980, 981, 1382, 5984, 2984, EDT 2001 or Pemulen TR2).

The polymers include, for example, polyacrylamides (Seppigel 305), polyvinyl alcohols, PVP, PVP/VA copolymers, polyglycols.

Preparations according to the invention in the form of emulsions comprise one or more emulsifiers. These emulsifiers can advantageously be chosen from the group of nonionic, anionic, cationic or amphoteric emulsifiers.

The nonionic emulsifiers include

a) partial fatty acid esters and fatty acid esters of polyhydric alcohols and ethoxylated derivatives thereof (e.g. glyceryl monostearates, sorbitan stearates, glyceryl stearyl citrates, sucrose stearates)

b) ethoxylated fatty alcohols and fatty acids

c) ethoxylated fatty amines, fatty acid amides, fatty acid alkanolamides

d) alkylphenol polyglycol ethers (e.g. Triton X).

The anionic emulsifiers include

a) soaps (e.g. sodium stearate)

b) fatty alcohol sulfates

c) mono-, di- and trialkylphosphoric esters and ethoxylates thereof.

The cationic emulsifiers include

a) quaternary ammonium compounds with a long-chain aliphatic radical, e.g. distearyldimonium chloride.

The amphoteric emulsifiers include

a) alkylamininoalkanecarboxylic acids

b) betaines, sulfobetaines

c) imidazoline derivatives.

In addition, there are naturally occurring emulsifiers, which include beeswax, wool wax, lecithin and sterols.

O/W emulsifiers can be advantageously chosen, for example, from the group of polyethoxylated or polypropoxylated or polyethoxylated and polypropoxylated products, e.g.:

fatty alcohol ethoxylates,

ethoxylated wool wax alcohols,

polyethylene glycol ethers of the general formula

R—O—(—CH₂—CH₂—O—)ₙ—R′,

fatty acid ethoxylates of the general formula

R—COO—(—CH₂—CH₂—O—)ₙ—H,

etherified fatty acid ethoxylates of the general formula

R—COO—(—CH₂—CH₂—O—)ₙ—R′,

esterified fatty acid ethoxylates of the general formula

R—COO—(—CH₂—CH₂—O—)ₙ—C(O)—R′,

polyethylene glycol glycerol fatty acid esters,

ethoxylated sorbitan esters,

cholesterol ethoxylates,

ethoxylated triglycerides,

alkyl ether carboxylic acids of the general formula

R—O—(—CH₂—CH₂—O—)ₙ—CH₂—COOH

and n are a number from 5 to 30,

polyoxyethylene sorbitol fatty acid esters,

alkyl ether sulfates of the general formula

R—O—(—CH₂—CH₂—O—)ₙ—SO₃—H,

fatty alcohol propoxylates of the general formula

R—O—(—CH₂—CH(CH₃)—O—)ₙ—H,

polypropylene glycol ethers of the general formula

R—O—(—CH₂—CH(CH₃)—O—)ₙ—R′,

propoxylated wool wax alcohols,

etherified fatty acid propoxylates

R—COO—(—CH₂—CH(CH₃)—O—)ₙ—R′,

esterified fatty acid propoxylates of the general formula

R—COO—(—CH₂—CH(CH₃)—O—)ₙ—C(O)—R′,

fatty acid propoxylates of the general formula

R—COO—(—CH₂—CH(CH₃)—O—)ₙ—H,

polypropylene glycol glycerol fatty acid esters,

propoxylated sorbitan esters,

cholesterol propoxylates,

propoxylated triglycerides,

alkyl ether carboxylic acids of the general formula

R—O—(—CH₂—CH(CH₃)O—)ₙ—CH₂—COOH,

alkyl ether sulfates or the parent acids of these sulfates of the general formula

R—O—(—CH₂—CH(CH₃)—O—)ₙ—SO₃—H,

fatty alcohol ethoxylates/propoxylates of the general formula

R—O—Xₙ—Yₘ—H,

polypropylene glycol ethers of the general formula

R—O—Xₙ—Yₘ—R′,

etherified fatty acid propoxylates of the general formula

R—COO—Xₙ—Yₘ—R′,

fatty acid ethoxylates/propoxylates of the general formula

R—COO—Xₙ—Yₘ—H.

According to the invention, particularly advantageous polyethoxylated or polypropoxylated or polyethoxylated and polypropoxylated O/W emulsifiers used are those chosen from the group of substances having HLB values of 11-18, very particularly advantageously having HLB values of 14.5-15.5, provided the O/W emulsifiers have saturated radicals R and R′. If the O/W emulsifiers have unsaturated radicals R and/or R′, or isoalkyl derivatives are present, then the preferred HLB value of such emulsifiers can also be lower or higher.

It is advantageous to choose the fatty alcohol ethoxylates from the group of ethoxylated stearyl alcohols, cetyl alcohols, cetylstearyl alcohols (cetearyl alcohols). Particular preference is given to:

polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20),

polyethylene glycol(13) isocetyl ether (isoceteth-13), polyethylene glycol(14) isocetyl ether (isoceteth-14), polyethylene glycol(1 5) isocetyl ether (isoceteth-1 5), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20),

polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-1 5),

polyethylene glycol(12) lauryl ether (laureth-12), polyethylene glycol(12) isolauryl ether (isolaureth-1 2),

polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol (19) cetylstearyl ether (ceteareth-19), polyethylene glycol(20) cetylstearyl ether (ceteareth-20).

It is also advantageous to choose the fatty acid ethoxylates from the following group:

polyethylene glycol(20) stearate, polyethylene glycol(21) stearate, polyethylene glycol(22) stearate, polyethylene glycol(23) stearate, polyethylene glycol(24) stearate, polyethylene glycol(25) stearate,

polyethylene glycol(12) isostearate, polyethylene glycol(13) isostearate, polyethylene glycol(14) isostearate, polyethylene glycol(15) isostearate, polyethylene glycol(16) isostearate, polyethylene glycol(1 7) isostearate, polyethylene glycol(1 8) isostearate, poly-ethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate,

polyethylene glycol(12) oleate, polyethylene glycol(13) oleate, polyethylene glycol(14) oleate, polyethylene glycol(1 5) oleate, polyethylene glycol(1 6) oleate, polyethylene glycol(17) oleate, polyethylene glycol(18) oleate, polyethylene glycol(19) oleate, polyethylene glycol(20) oleate.

The ethoxylated alkyl ether carboxylic acid or salt thereof which can be used is advantageously sodium laureth-11 carboxylate.

Sodium laureth 1-4 sulfate can be used advantageously as alkyl ether sulfate.

An advantageous ethoxylated cholesterol derivative which can be used is polyethylene glycol(30) cholesteryl ether. Polyethylene glycol(25) soyasterol has also proven successful.

Ethoxylated triglycerides which can be advantageously used are polyethylene glycol(60) Evening Primrose glycerides.

It is also advantageous to choose the polyethylene glycol glycerol fatty acid esters from the group polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate, polyethylene glycol(20) glyceryl oleate, polyethylene glycol(20) glyceryl isostearate, polyethylene glycol(1 8) glyceryl oleate/cocoate.

It is likewise favorable to choose the sorbitan esters from the group polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

Advantageous W/O emulsifiers which can be used are: fatty alcohols having 8 to 30 carbon atoms, monoglycerol esters of saturated and/or unsaturated, branched and/or unbranched alkanecarboxylic acids having a chain length of from 8 to 24, in particular 12-18, carbon atoms, diglycerol esters of saturated and/or unsaturated, branched and/or unbranched alkanecarboxylic acids having a chain length of from 8 to 24, in particular 12-18, carbon atoms, monoglycerol ethers of saturated and/or unsaturated, branched and/or unbranched alcohols having a chain length of from 8 to 24, in particular 12-18, carbon atoms, diglycerol ethers of saturated and/or unsaturated, branched and/or unbranched alcohols having a chain length of from 8 to 24, in particular 12-18, carbon atoms, propylene glycol esters of saturated and/or unsaturated, branched and/or unbranched alkanecarboxylic acids having a chain length of from 8 to 24, in particular 12-18, carbon atoms, and sorbitan esters of saturated and/or unsaturated, branched and/or unbranched alkanecarboxylic acids having a chain length of from 8 to 24, in particular 12-18, carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprate, glyceryl monocaprylate.

The examples below are intended to illustrate the invention, but not limit it. The numerals given refer to % by weight, unless stated otherwise.

### EXAMPLE 1

### O/W Cream

| | % by wt. |
|---|---|
| Glyceryl stearate citrate | 2.00 |
| Stearyl alcohol | 5.00 |
| Caprylic/capric triglycerides | 4.00 |
| Octyldodecanol | 4.00 |
| Glycerol | 3.00 |
| Carbomer | 0.10 |
| Carnitine | 1.00 |
| EDTA | 0.10 |
| Sodium hydroxide | q.s. |
| Preservative | q.s. |
| Perfume | q.s. |
| Water, demineralized | ad 100.00 |

pH adjusted to 6.0

### EXAMPLE 2

### O/W Cream

| | % by wt. |
|---|---|
| Glyceryl stearate citrate | 3.00 |
| Cetylstearyl alcohol | 3.00 |
| Paraffin oil | 2.00 |
| Caprylic/capric triglycerides | 4.00 |
| Dicaprylyl ether | 3.00 |
| Xanthan gum | 0.10 |
| Citric acid | 0.10 |
| Sodium citrate | 0.20 |
| Carnitine | 0.50 |
| Glycerol | 3.00 |
| Preservative | q.s. |
| Perfume | q.s. |
| Water | ad 100.00 |

pH adjusted to 5.5

### EXAMPLE 3

### O/W Cream

| | % by wt. |
|---|---|
| Glyceryl stearate | 4.00 |
| PEG-40 stearate | 1.00 |
| Cetyl alcohol | 3.00 |
| Caprylic/capric triglycerides | 5.00 |
| Paraffin oil | 5.00 |
| Glycerol | 3.00 |
| Carbomer | 0.10 |
| Carnitine | 0.25 |
| EDTA | 0.10 |
| α-Glucosylrutin | 0.05 |
| Sodium hydroxide | q.s. |
| Preservative | q.s. |
| Perfume | q.s. |
| Water, demineralized | ad 100.00 |

pH adjusted to 5.0

### EXAMPLE 4

### O/W Cream

| | % by wt. |
|---|---|
| Glyceryl stearate SE | 3.00 |
| Stearic acid | 1.00 |
| Cetyl alcohol | 2.00 |
| Dicaprylyl ether | 4.00 |
| Caprylic/capric triglycerides | 3.00 |
| Paraffin oil | 2.00 |
| Glycerol | 3.00 |
| Butylene glycol | 3.00 |
| Carbomer | 0.10 |
| Carnitine | 1.00 |
| Sodium hydroxide | q.s. |
| Preservative | q.s. |
| Perfume | q.s. |
| Water, demineralized | ad 100.00 |

pH adjusted to 7.0

### EXAMPLE 5

### O/W Cream

| | % by wt. |
|---|---|
| Polyglyceryl methylglucose distearate | 4.50 |
| Caprylic/capric triglycerides | 5.50 |
| Octyldodecanol | 4.50 |
| Cetylstearyl alcohol | 5.00 |
| Xanthan gum | 0.10 |
| Stearyl alcohol | 1.30 |
| Glycerol | 3.00 |
| Carnitine | 1.00 |
| EDTA | 0.10 |
| Preservative | q.s. |
| Perfume | q.s. |
| Water, demineralized | ad 100.00 |

pH adjusted to 5.5

### EXAMPLE 6

### O/W Lotion

| | % by wt. |
|---|---|
| Glyceryl stearate, Ceteth-20 | 1.00 |
| Sorbitan stearate | 1.00 |
| Stearyl alcohol | 1.00 |
| Caprylic/capric triglycerides | 2.00 |
| Paraffin oil | 4.00 |
| Glycerol | 3.00 |
| Carbomer | 0.10 |
| Acylcarnitine | 0.10 |
| Tocopherol | 0.05 |
| Sodium hydroxide | q.s. |
| Preservative | q.s. |
| Perfume | q.s. |
| Water, demineralized | ad 100.00 |

pH adjusted to 5.5

### EXAMPLE 7

### W/O cream

| | % by wt. |
|---|---|
| Lameform ® TGI | 3.50 |
| Glycerol | 3.00 |
| Dehymuls ® PGPH | 3.50 |
| Carnitine | 0.50 |
| Magnesium sulfate | 0.60 |
| Isopropyl stearate | 2.00 |
| Dicaprylyl ether | 8.00 |
| Cetearyl isononanoate | 6.00 |
| Preservative | q.s. |
| Perfume | q.s. |
| Water, demin. | ad 100.00 |

### EXAMPLE 8

### Emulsions Make-Up

| | % by wt. |
|---|---|
| Glyceryl stearate SE | 5.00 |
| Stearyl alcohol | 2.00 |
| Dimethicone | 2.00 |
| Glycerol | 3.00 |
| Carbomer | 0.15 |
| Mica | 1.00 |
| Magnesium silicate | 1.00 |
| Iron oxides | 1.00 |
| Titanium dioxide | 2.50 |
| Talc | 5.00 |
| Carnitine | 0.15 |
| Sodium hydroxide | q.s. |
| Preservative | q.s. |
| Perfume | q.s. |
| Water, demineralized | ad 100.00 |

pH adjusted to 6.0

### EXAMPLE 9

### W/O/W Cream

| | % by wt. |
|---|---|
| Glyceryl stearate | 3.00 |
| PEG-100 stearate | 0.75 |
| Behenyl alcohol | 2.00 |
| Caprylic/capric triglycerides | 8.0 |
| Octyldodecanol | 5.00 |
| C₁₂₋₁₅-alkyl benzoate | 3.00 |
| Panthenol | 3.00 |
| Butylhydroxytoluene | 0.05 |
| Magnesium sulfate (MgSO₄) | 0.80 |
| EDTA | 0.10 |
| Carnitine | 0.20 |
| Preservative | q.s. |
| Perfume | q.s. |
| Water, demineralized | ad 100.00 |

pH adjusted to 6.0

### EXAMPLE 10

### Hydrodispersion Gel

| | % by wt. |
|---|---|
| Carbomer | 0.40 |
| Xanthan gum | 0.20 |
| Cetylstearyl alcohol | 2.00 |
| C₁₂₋₁₅-alkyl benzoates | 5.00 |
| Caprylic/capric triglycerides | 3.00 |
| Glycerol | 3.00 |
| Carnitine | 0.20 |
| Sodium hydroxide | q.s. |
| Preservative | q.s. |
| Perfume | q.s. |
| Water, demineralized | ad 100.0 |

pH adjusted to 5.5

1. The use of carnitine and/or one or more acylcarnitines for producing cosmetic or dermatological preparations for increasing the biosynthesis of ceramide.

2. The use of carnitine and/or one or more acylcarnitines for producing cosmetic or dermatological preparations for improving the barrier properties of the skin.

3. The use of carnitine and/or one or more acylcarnitines for producing cosmetic or dermatological preparations for preventing or reducing skin drying.

4. The use as claimed in any of the preceding claims, characterized in that the preparations comprise 0.001-10% by weight of carnitine and/or one or more acylcarnitines, based on the total weight of the preparations.
